(19) United States
(12) Patent Application Publication
(10) Pub. No.: US 2006/0008866 A1
(43) Pub. Date: Jan. 12, 2006

(54) Apparatus and method for detecting human fecal contamination on hands and other objects using an illumination imaging device

(75) Inventors: Ricky Lee Flick, Merritt Island, FL (US); Gavin H. Poole, Fort Pierce, FL (US); Richard D. Stroman, Indian Harbor Beach, FL (US)

Correspondence Address: DENNIS L. COOK, ESQ.;THE LAW OFFICES OF DENNIS L COOK PLLC, 12718 DUPONT CIRCLE, TAMPA, FL, 33626, US

(73) Assignee: eMerge Interactive, Inc., Sebastian, FL (US)

(21) Appl. No.: 11/176,112

(22) Filed: Jul. 7, 2005

Related U.S. Application Data
(60) Provisional application No. 60/586,656, filed on Jul. 9, 2004.

(51) Int. Cl. C12Q 1/04; C12M 1/34

(52) U.S. Cl. 435/34; 435/288.7; 435/808; 250/459.1; 250/461.2

(57) ABSTRACT

According to the present invention there is disclosed a system and a method for detecting the presence of human fecal contamination on objects, such as a protein source, a worker's hands, work surfaces, tools or utensils. In one embodiment, there is included a supporting structure which supports a diffuse light source, the light source emitting light having a wavelength from the blue portion of the electromagnetic spectrum effective to elicit fluorescence from the fecal matter at wavelengths in the 600 nm to 800 nm range. A third wavelength of interest in the 600 nm to 800 nm range is also used as a reference control. Thereafter the wavelengths are combined in a series of mathematical steps to develop an algorithm or process to detect the presence of the human fecal material. There is also disclosed a method of using such a device to detect the presence of such contamination, optionally including further steps to identify the source of any contamination and to modify any practices so that the spread of contamination may be reduced.

### CROSS-REFERENCE TO RELATED APPLICATION

The present application claims the benefit of previously filed co-pending provisional patent application, Ser. No. 60/586,656 filed on Jul. 9, 2004.

### GOVERNMENT LICENSE RIGHTS

(Intentionally left blank)

### OBJECT OF THE INVENTION

An apparatus and method for detecting human fecal or other contamination on a user's hands or other objects using fluorescence spectroscopy is disclosed to help people fully wash their hands or other objects so as to assist in preventing contamination. The system is made up of four parts. First, there is an illumination system, by which the sample area of interest is irradiated with the excitation wavelength or wavelengths of light. The light source is such that the excitation wavelength or wavelengths are from the visible section of the electromagnetic spectrum, and thus would pose the least threat of injury to the subject. Secondly, there is a plurality of detectors, or a single detector capable of gathering data from multiple wavelengths, which are tuned to the emission wavelengths of interest. Next is a processing unit, where the emission responses from the different detectors are processed via a mathematical algorithm or algorithms. Finally, there is a visual display to indicate to the subject if there is contamination is on the sample surface.

### FIELD OF THE INVENTION

The invention relates to an improved method and apparatus for detecting human fecal or other contamination on a user's hands or a surface, or objects such as food products, cooking or cutting utensils, or hospital or medical facility tools or surfaces, by placing the objects or users hands in the inspecting area of the apparatus or scanning the apparatus over the object, using visible light fluorescent spectroscopy.

Microbial pathogens in food cause an estimated 6.5 million to 33 million cases of human illness and up to 9,000 deaths annually, according to the Council for Agricultural Science and Technology. Furthermore, the USDA Economic Research Service has recently reported that the annual cost of the food-borne illnesses caused by six common bacterial pathogens, Campylobacter spp., Clostridium perfringens, Escherichia coli 0157:H7, Listeria monocytogenes, Salmonella spp., and Staphylococcus aureus, ranges from 2.9 billion to 6.7 billion dollars (Food Institute Report, USDA, AER, December, 1996). The foods most likely to cause these illnesses are animal products such as red meat, poultry and eggs, seafood, and dairy products, and fresh fruits and vegetables.

Harmful microorganisms may be present in human feces which can be spread by contact and serve as sources for human diseases. The handling of food containing such contamination often causes the spread of these harmful microorganisms, typically by the contamination coming into contact with the hands or utensils used by food preparers. There is a need for a device to determine in real time if human fecal contamination is present on the hands or objects used by those working in the food service industry, or even in the home.

In addition, human fecal contamination can occur in different situations, as may be seen in the childcare, geriatric care, or healthcare industries, or many other places. In these industries, this contamination can similarly be spread by workers to other people, objects, surfaces, or to food items that are ingested by others. A device is needed in these and other contexts to assist workers in ensuring that contamination is not present, and where its presence is detected, ensuring that it is removed by thorough hand or object washing before it is spread.

Currently, there are a variety of complicated and time consuming methods available to determine whether fecal contamination is present on meat or other objects. These methods typically include human visual inspection, microbiological culture analysis, bioluminescent ATP-based assays, and antibody-based microbiological tests. These methods are not effective or efficient enough to use in real time to detect trace amounts of human fecal contamination on hands and other common objects used in the food processing and delivery industries.

Fluorescence spectroscopy has been commonly used for the analysis of a variety of compounds, microorganisms, and tissues. The use of fluorescence spectroscopy for the detection of contaminants on foods has also been previously disclosed. For example, Alfano (U.S. Pat. No. 5,474,910) disclosed a method and apparatus for detecting biological molecules and microorganisms by irradiating the sample material with UV light at a wavelength between about 250 to 325 nm and measuring the resultant fluorescence. Alfano further disclosed that the process could be used for detecting the bacterial spoilage of food products, including meat and poultry. Unlike the disclosure in Alfano, which teaches a method that detects biological molecules and microorganisms, the present invention instead teaches the detection of the presence of chlorophyll or other chemicals found in the host material (fecal material) in which the bacteria lives, and does so by irradiating a sample material with light at a wavelength between about 380 nm and 470 nm rather than 250 to 325 nm. More recently, Waldroup and Kirby (U.S. Pat. Nos. 5,621,215 and 5,895,521) disclosed a method and apparatus for detecting the contamination of meat or poultry with fecal material. As described therein, the meat or poultry is illuminated with UV light having a wavelength between about 320 to 420 nm, and examined for fluorescence.

There is also disclosed by Casey et al (U.S. Pat. No. 5,914,247) a method and apparatus for detecting fecal contamination on an animal carcass in near real-time using fluorescent spectroscopy. As taught therein, the surface of the carcass is illuminated with UV or visible light having a wavelength between 300-600 nm, and fluorescent light emissions having a wavelength between about 660 to 680 nm are then detected. The invention taught by Casey et al is useful for detection of ingesta and fecal contamination during the high speed processing of animal carcasses in a slaughterhouse, and is particularly adapted to be used within a short time after slaughter. The invention taught by Casey et al uses the fluorescence of the chlorophyll molecule to detect the ingesta or fecal matter on meat surfaces that may have been contaminated during the slaughter and processing of the carcass. However, the device described in the '247 patent is not compact, lightweight, portable, inexpensive, or suited to the spot-checking of the hands of workers, or the objects which may come into contact with fecal matter, but is instead directed at examination of meat products at high speeds in a slaughterhouse setting. Fortuitously the inventors of this disclosure have now discovered a method of detecting human fecal matter that would not necessarily contain a high level of chlorophyll.

Unfortunately, many of these prior art procedures and devices described above are either labor intensive, time consuming, insensitive, require large amounts of floor space, or are not cost-effective on a small scale, and thus are inappropriate for the food service, healthcare, child care or similar industries which may, in particular, allow exposure to human fecal contamination. Additionally, there are currently no known commercial, real-time systems available for detection of human fecal material and the potentially harmful bacteria that can be present in this fecal material. There is a need for an objective device that will give the retailer or commercial vendor the ability to ensure that its workers are not introducing or spreading contamination through their hands or their working utensils prior to the sale or serving of the food being handled. There is also a need for an objective device that can be used in the home or consumer environment to detect trace amounts of fecal matter on human hands.

Accordingly, there is a need for a cost-effective device, which can quickly, objectively, and accurately be used to detect whether workers hands or other objects contain human fecal contamination at locations such as at grocery stores and restaurants.

There is also a need for a device and method that the meat processing and grocery industry can rely upon to objectively determine that their workers hands are free of contamination. Such a device and method may be incorporated into a routine quality control process or integrated into an employee identification scheme.

There is also a need for a device, which can safely, quickly and accurately detect the presence of human fecal matter on objects used by the workers. For example, in the food services, healthcare, childcare, assisted living, consumer households and other industries which allow for exposure to human fecal contamination, there is a need for a device, which can quickly, accurately and objectively detect whether human fecal matter is present on any object which may come into contact with human fecal contamination. These objects may include workers hands as well as endoscopes, knives or other surgical tools used in the healthcare industry as well as food preparation devices or surfaces that food may come into contact with.

There is also a need for a device that provides for a real-time method of determining whether contamination is present, such that the spread of such contamination may be reduced. Such a device would also allow workers to take steps to remove such contamination before it is spread to other people or objects, and further to adjust their practices to prevent such contamination in the future. In order to be useful to food preparation industry, or the childcare and healthcare or consumer markets, such a device would also have to be affordable and small enough to fit within a minimal amount of space. There is also a need for a device that may be made available to employees for use prior to their returning to work after using the restroom such that contamination may not be spread.

There is also a need for a device, which serves the purposes listed above, and which also allows for hands-free operation or is portable and can easily be moved to the object to be inspected.

There is also a need for a device, which can objectively identify any fecal contamination on the hands of employees that can be integrated into an employee identification system. This system can also be used to screen workers prior to entering the workplace or beginning their shift. This system can be used as an integral part of an overall quality control or Hazardous Analysis Critical Control Point (HACCP) program or other system.

### SUMMARY OF THE INVENTION

We have now invented a novel and improved method and apparatus for detecting human fecal contamination on the surface of a user's hands or an object using visible light fluorescent spectroscopy. According to the present invention, there is disclosed a system, which allows a hand or other object to be placed in a designated spatial area and illuminated with visible light emitted by a light source. The system would be made up of four parts. First, there is an illumination system, by which the sample area of interest is radiated with the excitation wavelength or wavelengths of light. The light source is such that the excitation wavelength or wavelengths are from the visible section of the electromagnetic spectrum, and thus would pose the least threat of injury to the subject. Secondly, there is a plurality of detectors, or a single detector capable of gathering data from multiple wavelengths which would be tuned to the emission wavelengths of interest. Next would be a processing unit, where the emission responses from the different detectors are processed via a mathematical algorithm or algorithms. Finally, there is a visual display to indicate to the subject if contamination is on the sample surface. The processor or CPU may also process the signal from the detector and transmit it to a result indicator tracking system, an external network, employee ID reader, or any combination thereof A proximity sensor may also be optionally used to ensure that an object is present in the area adjacent to the system of the present invention before the light source will be activated.

In accordance with this discovery, it is an object of this invention to provide a fixed or portable apparatus for real-time detection of human fecal contamination on the surface of human hands or the objects they use to handle and prepare food.

It is also an object of the present invention to provide a fixed or portable device which may be used to detect human fecal matter on the hands of workers in various industries, as may arise from a variety of sources. Once such contamination is identified, steps may be taken to remove such contamination before it is spread. It is intended that such industries as meat processing, healthcare, childcare, and food sellers and preparers could use such a device.

It is also an object of the present invention to provide a fixed or portable apparatus that may additionally be used to inspect meat products such as such as beef, lamb, pork, chicken, turkey and the like, and the tools used to process them, for human fecal contamination.

It is also an object of the present invention to provide a fixed or portable apparatus and method to identify such contamination and then to take steps to modify the workers' activities or processes to prevent such contamination in the future.

Another object of the present invention is to provide safety features, which minimize the likelihood of a user being exposed to the light emitted.

It is an object of the present invention to provide a device and method that the food preparation industry and consumers can rely upon to certify that their food is free of human fecal contamination. Such a device and method may be incorporated into a routine quality control process, or as part of a HACCP or other food safety program.

Another object of the present invention is to provide a device, which can objectively identify any fecal contamination on the hands of employees that can be integrated into an employee identification system. This system can also be used to screen workers prior to entering the workplace, beginning their shift, or returning from the restroom. This system can be used as an integral part of an overall quality control or Hazardous Analysis Critical Control Point (HACCP) program or other system.

Another object of the present invention is to provide an integrated imaging system, which can provide additional sensitivity over utilizing the human eye to detect the fluorescence of the human fecal material. Yet another object of the present invention is to provide an apparatus which, meets the above needs, is affordable and small enough to fit within a minimal amount of space, or is portable.

Other objects and advantages of the invention will become apparent from the ensuing description.

### BRIEF DESCRIPTION OF THE DRAWINGS

The following figures set forth preferred embodiments the present invention:

FIG. 1 is a perspective view of a preferred embodiment of the present invention.

FIGS. 2a and 2b are perspective views of a preferred embodiment in use, illuminating a user's hands.

FIGS. 3 and 4 show a front and side view of the configuration of the components within a preferred embodiment of the present invention, including: a light source or illumination system that illuminates an area adjacent to the system; a filter; a detection system; and an LCD or monitor display;

FIG. 5 shows an algorithm used to detect human fecal matter.

### DETAILED DESCRIPTION OF THE INVENTION

The process and apparatus of this invention may be used for detecting the human feces that may be present on the surface of a human's hands. It may also be used for detection of human fecal matter on other objects, including on cuts of meat of wild or domestic meat producing animals, including but not limited to mammals and birds such as bovine, poultry, porcine, ovine, caprine, equine, and ratites.

The apparatus may also be used on objects used for food handling, including knives, cleavers, cutting boards, counter tops and the like, or on medical implements or surfaces. The apparatus may also be used to detect the presence of human fecal contamination on the bodies of workers who may come into contact with such fecal matter.

In practice, the area of interest (for example the hands) would be illuminated with light from the blue portion of the electromagnetic spectrum or as used in the preferred embodiment, the excitation wavelength of 400-440 nm. Unlike the emission spectra of meat producing animals' fecal material, the emission or fluorescence response wavelengths of human fecal material is much more complex, due to a higher degree of variability in the diet of humans over animals. Thus, looking for a marker pigment such as chlorophyll (as was disclosed in U.S. Pat. No. 5,914,247) is not feasible, since not all humans will eat the same things (for example, certain low carbohydrate/high protein diets would severely limit the amount of chlorophyll and chlorophyll by-products found in human feces). Thus a plurality of markers need to be used.

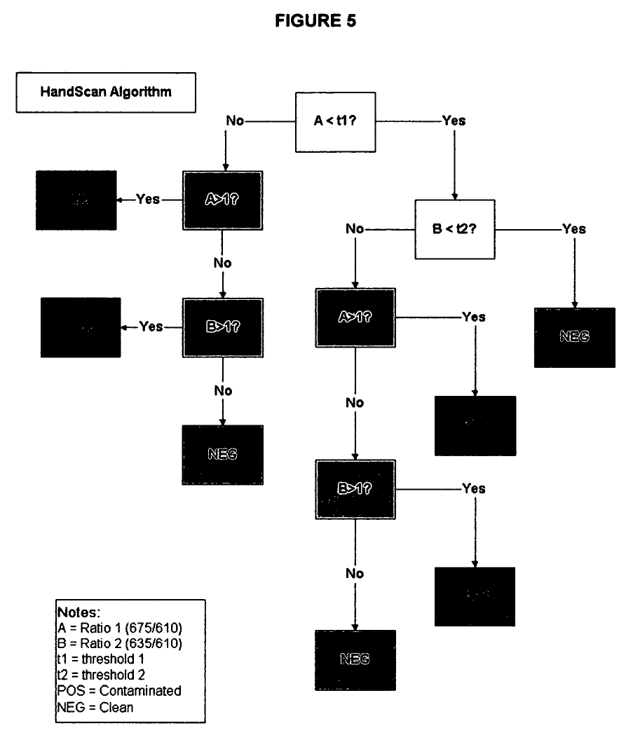

With multiple marker chemicals, multiple emission wavelength peaks are returned. Currently, as disclosed in this description of one embodiment there are two emission peaks in the range of 600 nm to 800 nm that have been identified as useful in differentiating fecal material from human skin—approximately 675 nm and 635 nm. A third wavelength of interest is also used in this one embodiment as a reference control—610 nm. With these multiple wavelengths, there is a more complex method of differentiating fecal material from the sample area of interest. Instead of looking at the existence and amplitude of a single peak at one wavelength, in this embodiment the 3 wavelengths are combined in a series of mathematical steps to develop an algorithm or process to find the fecal material. The structure of an example of the human fecal detection algorithm as based on the wavelengths identified above is shown in FIG. 5. This figure is only one example of how an algorithm might be used, and is not intended to be limiting. Other variations of this algorithm might also be useable, and could be developed by someone knowledgeable in the ways of algorithm development.

### EXAMPLE 1

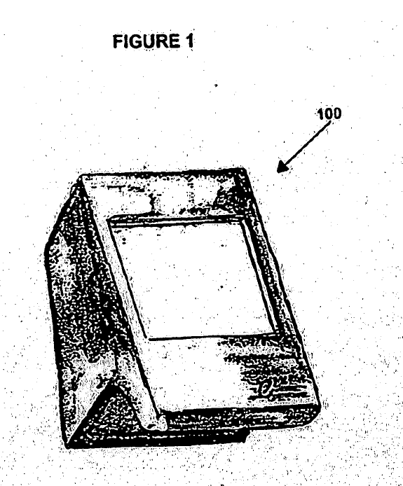

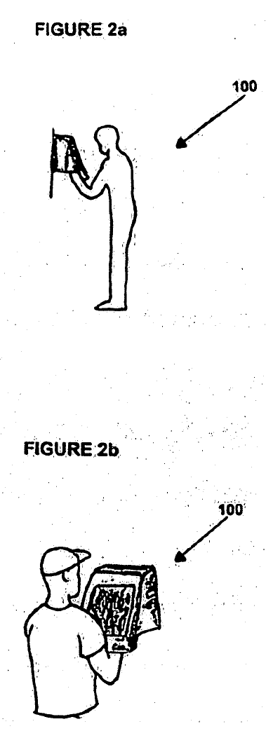

One embodiment of the present invention 100 can be seen with reference to FIGS. 1-5. A perspective view of such an embodiment 100 is shown in FIG. 1. FIGS. 2a and b depict a typical user employing an embodiment of the present system 100 to inspect his hands, or any other object, for contamination. It is to be understood that the word “object” as used in this Specification is meant to include both a person's hands or body and non-skin items, such as utensils, work surfaces or tools, or even meat products.

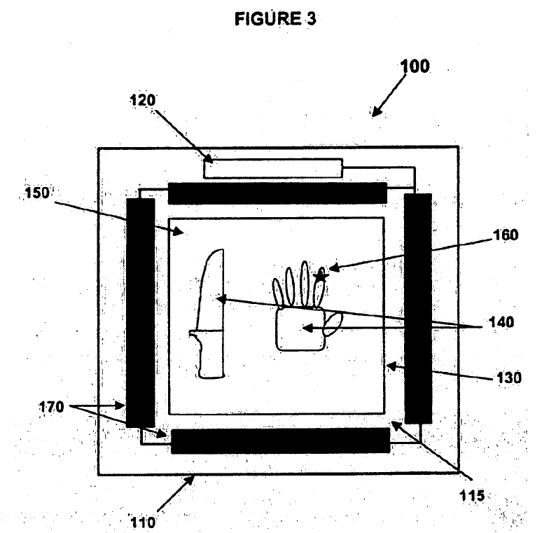

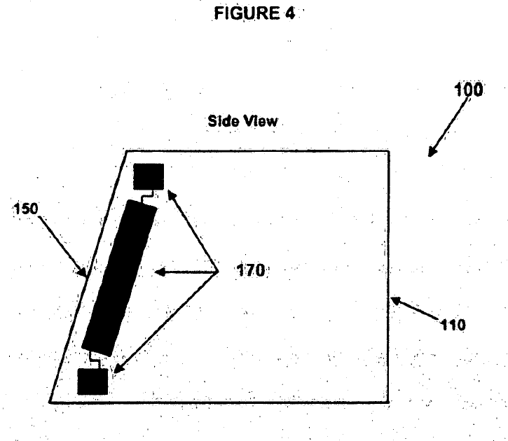

The configuration of such an embodiment is shown in FIGS. 3 and 4, and includes a mounting structure 110 supporting a light source 120 capable of generating light in the visible range. Emission light from light source 120 can be created by an array of LED's, mercury vapor lights, fluorescent lamps any other source well-known to those in the art to be capable of generating light in the appropriate range. In one embodiment, the light is emitted and positioned so as to permit illumination of the object of interest when the object is placed in the illuminated inspection area 115 adjacent to the system 100 of the present invention.

The system 100 may be mounted on a wall or other supporting device leaving an inspection area 115 at least large enough for an object 140 to be examined for contamination or be portable for easy inspection of objects that are unable to me moved or placed within the inspection area of a wall mounted system. These embodiments also include detection devices 170 such as photo detectors or CCDs sensitive to the wavelengths identified above. Without being limited thereto, suitable photo detectors for use herein include photodiode detectors, photomultipliers, amplifiers or image intensifiers, CCD cameras, and photo cathodes and micro channel plates (i.e. “Night vision” technology). One or more optical filters 130 are preferably positioned between the inspection area 115 and the photo detectors 170 to selectively transmit light in the range of interest, while preventing transmission of back-scattered excitation light. Filters are preferably effective to remove wavelengths of light less than about 600 and greater than about 800 nm.

Additionally, not shown in the figures, a processor, such as a CPU, or a programmable logic device, could control the operation of the system, including receiving signals from an activation device, such as a user keypad. The processor receives signals from the detection devices 170 and transmits them to a result indicator after the wavelengths are combined in a series of mathematical steps as shown in FIG. 5 to develop an algorithm or process to find the fecal material. The findings are then displayed on a display monitor 150, or an external network or employee ID reader, or any combination thereof. The entire system is powered by a power source (not shown), such as a battery for a portable device or power cord connected to a utility for the wall mounted device.

Also, a proximity sensor (not shown) may also be optionally used to ensure that an object is in fact present in the inspection area 115 adjacent to the system 100 of the present invention before the light source will be activated. Specifically, such object sensing device or proximity sensor may provide for electronic control of the light emissions, such that light will not be emitted from the light source unless an object to be examined is within the inspection area 115, or alternatively, within a certain specified distance from the light source. Such use of a distance sensor and affiliated circuitry provides for increased safety to the user and others in the vicinity in that the device will not be activated except when there is an object 140 present in the inspection area 115 adjacent to the system, thus providing the increased benefit of reducing any unintended and thus unnecessary exposure to the light source 120. Object sensing technologies appropriate for such an embodiment would be well-known to those in the distance-sensing art and would include, by way of example only, infrared and ultrasonic proximity-sensing or photo-electric technologies. The circuitry for creating such an object safety mechanism is also well known in the art.

In an alternative embodiment, the output signal from the photo detectors 170 may be relayed to a recording instrument (not shown), such as an oscilloscope, desktop computer, hard drive, printer or any other device known in the art for presenting or storing a graphical display of fluorescent spectra intensity.

In another alternative embodiment, the photo detectors 170 may be in communication with the processor, which is in turn in communication with a cleaning/disinfection indicator when the fluorescent intensity at the desired ranges has exceeded a predetermined threshold value. Signals may include for example, audible alarms, visible lights or LEDs, or any combination of the above. Thus, as taught herein, the present invention may aid in objectively identifying the presence of contaminant.

The operation of the preferred embodiment of the present invention can also be appreciated with respect to FIGS. 3 and 4. In operation, the light source 120 that emits light in the blue spectrum, or as used in the preferred embodiment, the excitation wavelength of 400-440 nm, is activated when the user puts an object 140, such as a hand or an instrument, into the inspection area 115. If human fecal matter 160 is present there are emission peaks that have been identified as useful in differentiating fecal material from human skin in the 600 nm to 800 nm range, or in the preferred embodiment, two peaks of approximately 675 nm and 635 nm. A third wavelength of interest in the 600 nm to 800 nm range is also used as a reference control, or in the embodiment discussed above, 610 nm. Thereafter the 3 wavelengths are combined in a series of mathematical steps as shown in FIG. 5 to develop an algorithm or process to determine its presence and indicate such on the display 150.

Upon detection of fecal contamination, the user's hand or other object 140 may be washed, disinfected or otherwise treated to remove feces 160 from the surface thereof. The process for detecting feces on the washed surface is then repeated, followed by additional washing and/or decontamination steps if necessary, until all traces of feces 160 have been removed or destroyed. A variety of washing solutions and disinfectants are known in the art and are suitable for use herein and include but are not limited to pressurized water or steam sprays, organic acids, chlorine, inorganic acids, detergents and treatment with radiation, most of which are not suitable for use other than on inanimate objects. Once the user's hands or other object 140 has been determined to be free of contamination 160 as evidenced by the lack of fluorescence at the described range, the object 140 may be returned to standard use or prepared for consumption, in the case of food products.

The detection of fecal contamination 160 on the object 140 also allows the user to adjust and improve their work practices in order to prevent contamination wherever possible and increase sanitation. When the present invention is used on meat, an additional benefit is an improvement in meat quality.

In yet another embodiment, the system 100 can be connected to an employee identification system for monitoring cleanliness after hand washing or handling of potentially contaminated meat products. Such a system 100 may additionally be operated in a “wireless” mode to update a central database on a given employee's cleanliness prior to entering the workplace. By way of example, the use of a photo detector and recording mechanism may be useful for the detection of human fecal matter on the hands of workers at a restaurant, such as when they first report for work or at various times throughout the day. Such a system would be valuable to audit the cleanliness of workers and keep records of those tests.

Such a system 100 may also be used as part of a method for reducing the spread of fecal contamination by a worker from an object to other items in a workplace. Again, such objects would include the hands of workers in the food processing or food handling industries, or any worker who handles meat and non-meat objects, which may potentially be contaminated. Such objects would also include the hands of such workers at the time they report for work, after bathroom break, or any time throughout the course of the day.

Such a method for reducing the spread of contamination would include first placing the object into an area adjacent to the present system. This step would occur at least one time per day, and ideally would occur periodically throughout the day, including when the worker first reports to work, after bathroom breaks, or at random or periodic intervals. Once the object 140 is placed in the adjacent area 115, the object 140 would be illuminated with light in the blue spectrum having a wavelength effective to elicit fluorescence in the 600 nm to 800 nm range, and more particularly in one embodiment, at the two emission peaks that have been identified as useful in differentiating fecal material from human skin approximately—675 nm and 635 nm. A third wavelength in the 600 mn to 800 nm range of interest is also used as a reference control. In this one embodiment that third wavelength is 610 nm. Thereafter the wavelengths are combined in a series of mathematical steps as shown in FIG. 5 to develop an algorithm or process to determine the presence of the fecal material.

To maximize the usefulness of this process, the results of the detecting step could be preserved for later review. By reviewing the preserved results, the user could identify trends relating to contamination, and thus take corrective action based upon these trends to reduce the instances of continued handling of contaminated objects. Such corrective actions could include anything from cleaning the contaminated object before returning to the workstation, to implementing required cleaning steps at various stages or times.

It is understood that the foregoing detailed description is given merely by way of illustration and that modifications and variations may be made therein without departing from the spirit and scope of the invention.

What is claimed is:

1. A system for detecting the presence of human ingesta or human fecal matter on the surface of an object that is placed in an inspection area, the system comprising:
a) a fixed or portable supporting structure;
b) a light source;
c) said supporting structure supporting said light source;
d) said light source emitting light into the inspection area;
e) said emitted light in the visible light spectrum having one or more wavelengths effective to elicit fluorescence from the human ingesta or human fecal matter on the object in the 600 nm to 800 nm range;
f) one or more light detection devices;
g) said one or more light detection devices being positioned so as to detect fluorescence light emissions from the inspection area;
h) said one or more light detection devices detecting fluorescent light emissions having a wavelength between about 600 to 800 nm from the object surface;
i) a processor;
j) an indicator;
k) said processor being in communication with said one or more light detection devices; and,
l) said processor transmitting a signal between said one or more light detection devices and said indicator when said one or more light detection devices detect fluorescent light emissions of two or more significant wavelengths.

2. The system of claim 1, wherein:
said light source emits light at wavelengths between about 400-440 nm.

3. The system of claim 2 where said light source is a laser, an array of LEDs, a mercury vapor light source, or fluorescent lamps.

4. The system of claim 4 where said one or more light detection devices are photodiode detectors, phototransistors, photomultipliers, amplifiers, image intensifiers, CCD cameras, CMOS cameras, photocathodes, or microchannel plates.

5. The system of claim 1 further comprising a viewing lens or display screen in communication with said processor.

6. The system of claim 1 further comprising a recording instrument in communication with said processor.

7. The system of claim 1 wherein said recording instrument includes an oscilloscope, a desktop computer, a hard drive, or a printer.

8. The system of claim 1 wherein said indicator is an audible alarm, visible lights, LEDs, or any combination thereof.

9. The system of claim 1 further comprising a means for detecting the presence of an object to be examined within the inspection area.

10. The system of claim 9, further comprising:
a) a means for preventing said light source from emitting light; and,
b) wherein said means for preventing prevents said light source from emitting light unless said means for detecting detects the presence of an object within the area adjacent to the system.

11. The system of claim 10, wherein said means for detecting is infrared proximity sensing, ultrasound proximity testing, mechanical switch or photo-electric devices.

12. The system of claim 1 further including one or more optical filters placed between the object that is placed in the inspection and said one or more light detection devices wherein said one or more optical filters are capable of removing wavelengths of light less than about 600 nm and greater than about 800 nm.

13. The system of claim 1 wherein said processor compares fluorescent intensities of peaks of interest against a control wavelength intensity.

14. A system for detecting the presence of human ingesta or human fecal matter on the surface of an object that is placed in an inspection area, the system comprising:
a) a fixed or portable supporting structure;
b) a light source;
c) said supporting structure supporting said light source;
d) said light source emitting light having one or more wavelengths of about 400-440 nm into the area adjacent to the system;
e) one or more light detection devices;
f) said one or more light detection devices being positioned so as to detect fluorescence light emissions from the area adjacent to the system;
g) said one or more light detection devices detecting fluorescent light emissions having two or more peak intensity wavelengths between about 600 to 800 nm from the object surface.
h) one or more light filters;
i) said one or more light filters being positioned between said light detection device and the area adjacent to the system;
j) said one or more light filters substantially filtering out light other than light at a wavelength between about 600 to 800 nm;
k) aprocessor;
l) an indicator;
m) said processor being in communication with said one or more detection devices;
n) said processor being capable of comparing fluorescent wavelength peaks of interest against a control wavelength intensity; and,
o) said processor transmitting a signal between said light detection device and said indicator when said wavelength peaks of interest are properly compared against said control wavelength such as to indicate the presence of human ingesta or human fecal matter.

15. The system of claim 14 further comprising a means for detecting the presence of an object to be examined within the area adjacent to the system.

16. The system of claim 15, further comprising:
a) a means for preventing said light source from emitting light; and,
b) wherein said means for preventing prevents said light source from emitting light unless said means for detecting detects the presence of an object within the area adjacent to the system.

17. The system of claim 16, wherein said means for detecting includes infrared proximity sensing, ultrasound proximity testing, or photo-electric testing.

18. A method for detecting the presence of human ingesta or human fecal matter on the surface of an object using a system, where the system has is portable or is mounted on a support device such as a wall and has a supporting structure and a light source which emits light having a wavelength effective to elicit fluorescence from the human ingesta or human fecal matter on the object at two or more wavelengths between about 600 to 800 nm, comprising the steps of:
a) placing the object in an inspection area;
b) illuminating the object in the inspection area with light having a wavelengths effective to elicit fluorescence from the human ingesta and/or human fecal matter at two or more wavelengths between 600 to 800 nm; and
c) detecting fluorescent light emissions from the surface of the object at two or more wavelengths between about 600 to 800 nm, wherein detection of significant fluorescent light emissions at said two or more wavelengths between about 600 to 800 nm is an indication of the presence of human ingesta or human fecal material on said surface.

19. The method of claim 18 wherein the method further comprises washing or decontaminating the object when said detecting step results in any fluorescent light emission from said object at two or more wavelengths between about 600 to 800 nm being detected.

20. The method of claim 19 wherein said washing or decontaminating step is performed using a wash solution including but not limited to pressurized water, steam, organic acids, chlorine, inorganic acids, and detergents, or any combination thereof.

21. The method of claim 20 further comprising repeating said illuminating and detecting and washing or decontaminating steps until no significant fluorescent light emission having two or more wavelengths between about 600 to 800 nm is detected.
